(19) United States
(12) Patent Application Publication
(10) Pub. No.: US 2023/0380994 A1
(43) Pub. Date: Nov. 30, 2023

(54) Powered Joint System with Enhanced Neural-Based Controller

(71) Applicant: UNIVERSITY OF UTAH RESEARCH FOUNDATION

(72) Inventors: Tommaso LENZI, Salt Lake City, UT (US); Grace HUNT, Salt Lake City, UT (US)

(21) Appl. No.: 18/032,790

(22) PCT Filed: Oct. 20, 2021
(86) PCT No.: PCT/US2021/055893

§ 371 Date: Apr. 19, 2023

Related U.S. Application Data
(60) Provisional application No. 63/094,222, filed on Oct. 20, 2020.

(51) Int. Cl. A61F 2/72; A61F 2/64; A61F 2/70; A61F 2/66

(52) U.S. Cl. CPC A61F 2/72; A61F 2/64; A61F 2/70; A61F 2/6607; A61F 2002/701; A61F 2002/704

(57) ABSTRACT

A powered joint system for providing volitional control of joint movement includes a knee joint, one or more electromyography (EMG) sensors, and a controller. The one or more EMG sensors are adapted for placement on skin of a residual limb of a user to detect EMG signals from a posterior side of a residual limb. The controller is communicatively coupled to the knee joint and the one or more EMG sensors. The controller comprises one or more processors and one or more hardware storage devices storing instructions that are executable by the one or more processors to configure the controller to perform various acts, including to receive an EMG signal from the one or more EMG sensors (the EMG signal being representative of muscle activation at the posterior side of the residual limb of the user) and determine a target knee torque based on the EMG signal.

### CROSS-REFERENCE TO RELATED APPLICATIONS

This application claims priority to U.S. Provisional Patent Application Ser. No. 63/094,222, filed Oct. 20, 2020 and titled “Powered Prosthesis with Enhanced Neural-Based Controller”, the entirety of which is incorporated herein by this reference.

### STATEMENT REGARDING FEDERALLY SPONSORED RESEARCH

This invention was made with government support under grant no. HD098154 awarded by the National Institutes of Health and grant no. 1925371 awarded by the National Science Foundation. The government has certain rights in this invention.

### BACKGROUND

Above-knee amputation disrupts the natural coordination of biological legs, limiting the mobility of individuals with amputations. After above-knee amputation, the thigh muscles are severed from their attachment points below the knee. The knee and ankle joints are replaced by passive prosthetic joints that cannot perform the biomechanical functions of the missing biological leg joints. Individuals with amputations must rely on their intact leg and upper body to compensate for the limitations of the prosthesis, resulting in slower, less stable, and less efficient ambulation. Compensatory reliance by amputees on their intact leg and upper body often leads to secondary physical conditions such as back pain, osteoarthritis, and/or osteoporosis. The limited functional mobility provided by available prostheses severely affects the quality of life of millions of individuals world-wide.

Powered prostheses present a promising solution to this problem. In contrast to conventional devices, powered prostheses have battery-operated servomotors that can generate the torque and power necessary to imitate the biomechanical function of the missing biological leg. Appropriate controllers are used to synchronize the movements of the prosthesis with the user's neuromuscular system. A common approach to powered prosthesis control is to identify the user's intended activity, such as standing up or walking, and then impose a fixed, pre-planned prosthesis action that imitates the behavior of an intact biological leg during the intended activity. Using this approach, powered prostheses have shown the ability to assist individuals with above-knee amputations in structured laboratory environments. However, the real world is highly variable. Timely and accurate classification of all possible variations of each ambulation activity is both challenging and critical—any misclassification of the user's intended movement can cause the prosthesis to perform a different activity than the user expects, increasing the likelihood of falls and injuries. Moreover, every activity typically requires a dedicated controller to adapt to variations due to the subject preference or the variability of the environment.

Accordingly, there is an ongoing need for improved controller systems for powered prostheses.

The subject matter claimed herein is not limited to embodiments that solve any disadvantages or that operate only in environments such as those described above. Rather, this background is only provided to illustrate one exemplary technology area where some embodiments described herein may be practiced.

### SUMMARY

Disclosed embodiments include a powered joint system that is configured to provide volitional control of powered joint movement. The powered joint system includes a knee joint, ankle joint, one or more electromyography (EMG) sensors, and a controller. The one or more EMG sensors are adapted for placement on skin of a residual limb of a user to detect EMG signals from a posterior side of a limb (e.g., a residual limb). The controller is communicatively coupled to the knee joint, ankle joint, and the one or more EMG sensors. The controller comprises one or more processors and one or more hardware storage devices storing instructions that are executable by the one or more processors to configure the controller to perform various acts, including to receive an EMG signal from the one or more EMG sensors (the EMG signal being representative of muscle activation at the posterior side of the limb of the user) and determine a target knee and ankle behavior based on the EMG signal.

This summary is provided to introduce a selection of concepts in a simplified form that are further described below in the detailed description. This summary is not intended to identify key features or essential features of the claimed subject matter, nor is it intended to be used as an indication of the scope of the claimed subject matter.

### BRIEF DESCRIPTION OF THE DRAWINGS

In order to describe the manner in which the above-recited and other advantages and features can be obtained, a more particular description of the subject matter briefly described above will be rendered by reference to specific embodiments which are illustrated in the appended drawings. Understanding that these drawings depict only typical embodiments and are not therefore to be considered limiting in scope, embodiments will be described and explained with additional specificity and detail through the use of the accompanying drawings.

FIG. 1 illustrates a conceptual representation of operation of a shared neural controller, according to the present disclosure;

FIG. 2 illustrates a perspective view of example components of an example powered knee and ankle prosthesis;

FIG. 3 illustrates an example flow diagram depicting acts associated with providing volitional control of prosthesis joint movement, in accordance with the present disclosure;

FIG. 4 illustrates an example experimental implementation of a powered prosthesis with a shared neural controller;

FIG. 5A illustrates an anterior view of example placement of an electromyography electrode on an intact limb of a user;

FIG. 5B illustrates a posterior view of example placement of an electromyography electrode on a residual limb of a user;

FIG. 6 illustrates example graphs showing comparisons between stand-up with powered prosthesis under the disclosed shared neural controller vs. standard of care (passive prosthesis);

FIG. 7 illustrates example graphs showing time-series of residual limb biceps femoris EMG signals and the joint angles, joint torques, and joint powers at the prosthesis knee and prosthesis ankle joint during each activity;

FIG. 8 illustrates graphs indicating analysis of the knee torque as a function of the EMG activation;

FIG. 9 illustrates cartesian positions of body segments during a circuit of activities including standing up, walking, squatting, turning, walking, lunging, walking, and sitting down;

FIG. 10 illustrates a graph of the knee and ankle angle during a circuit of activities including standing up, walking, squatting, turning, walking, lunging, walking, and sitting down; and

FIG. 11 illustrates a comparison of walking with the powered knee ankle prosthesis under the disclosed shared neural controller compared to walking with passive prostheses.

### DETAILED DESCRIPTION

### Overview

Standing up with conventional passive prostheses is challenging because of the inability of the passive prosthesis joints to provide positive assistive forces. A powered prosthesis can address this problem by actively generating torque as needed. However, proper synchronization of the powered prosthesis with the user's neuromuscular system is necessary to effectively assist the user. Synchronization is particularly challenging in the real world, due to the high variability of the environment.

Powered prosthesis controllers commonly aim to identify the user's intended activity and impose a fixed, pre-planned prosthesis action that imitates the movement of biological limbs during that activity. There are considerable limitations to the viability of this approach for real-world implementation. For example, the classification algorithms used to identify the subject's intended activity often must be trained using subject-specific, labelled data. Furthermore, the classification accuracy typically decreases over time, and retraining can be difficult and unsafe for the user to perform at home, requiring further intervention and participation by trained personnel.

When a conventional powered prosthesis controller is used, even a single misclassification can cause a misstep, which may result in a fall and/or injury. Moreover, every activity and every variation thereof typically requires a dedicated controller. Each of these controllers must be trained or manually tuned for each subject, which is costly, time consuming, and requires expertise not commonly available to clinicians.

Significant effort has been made to improve powered prosthesis controllers, primarily focusing on the problem of classification. Computer vision and range sensors also have the potential to improve classification accuracy. However, real-world implementation of computer vision and range sensors is associated with many challenges, such as camera placement, privacy, societal acceptance, etc.

Utilizing electromyography (EMG) from residual-limb muscles has the potential to improve classification accuracy, compared to using mechanical sensors alone. For example, neural control of a powered knee prosthesis has been combined with conventional state-determined knee impedances during stair ascent and walking. However, co-activation of the residual-limb muscles presents a key limitation to the viability of this antagonist approach for weight bearing activities. Classification-based EMG controllers can, at best, match the performance of non-neural controllers, while still requiring extensive controller training, subject-specific tuning, and intensive, multi-week, multi-session subject training. These obstacles reduce the clinical viability of classification-based EMG controllers.

Regardless of the specific sensors used, training classification algorithms of conventional powered prosthesis controllers requires the user to perform multiple repetitions of each activity as well as the transitions between activities, which can be taxing and even dangerous for the user without the supervision of trained personnel. Most importantly, even with perfect classification, every possible variation of each activity requires a separate, pre-tuned controller. Thus, powered prosthesis controllers based on any activity classification scheme have fundamental limitations that hamper their safety and usability in real-world implementations.

The present disclosure presents a fundamental departure from existing powered prosthesis controller paradigms. Rather than aiming to improve classification of the user's intended activity, disclosed embodiments can be implemented to give the user volitional control over the powered prosthesis using neural commands from the residual limb. For example, the present disclosure shows, among other things, that a shared neural controller that combines neural signals from a single hip extensor muscle with robot control enables standing up, squatting, lunging, and walking without explicit classification of the user's intended activity, controller tuning, or subject training.

In non-amputee individuals, knee extension torque is provided by the quadriceps muscle. Thus, it may seem logical to use the EMG signals produced by a quadricep muscle to drive the knee extension torque generated by a powered prosthesis. However, after above-knee amputation, the quadricep muscles lose their knee extension function. The vastus muscles atrophy and the rectus femoris becomes a monoarticular hip flexor. The present disclosure shows that the biceps femoris, a biarticular hamstring muscle in nonamputee individuals, provides a viable alternative to drive the knee extension torque generated by a powered prosthesis. After above-knee amputation, the biceps femoris loses its knee flexion function, but its hip extension function is retained. The biceps femoris is naturally active during standing up, when both hip extension torque and knee extension torque are required to counteract gravity. The biceps femoris is also naturally active during the stance phase of walking, when hip extension torque is necessary to propel the body forward and upward and knee extension torque is necessary to prevent the knee from collapsing. Because the biceps femoris naturally activates when knee extension torque is necessary, such as during standing up and during the stance phase of walking, users do not need to learn a new muscle activation pattern to use the disclosed shared neural controller. When flexion torque is required, as in the swing phase of walking, robot control may be utilized in the form of an indirect volitional control that automatically adapts the prosthesis trajectory based on the movements of the user's residual limb. Such functionality enables users to modulate the foot clearance while walking and crossing over obstacles without explicit classification of the environment. Thus, the EMG signal from the biceps femoris provides an intuitive input to provide direct volitional control of the powered prosthesis during movements that require knee extension torque, and the robot control provides indirect volitional control during movements that require flexion torque. Furthermore, EMG signal may be used to modify prosthesis behavior in existing controllers by multiplying or adding terms based on EMG signals. This allows the user to modify the behavior of the prosthesis when using existing controllers, in order to facilitate transitioning between activities such as walking and stairs. In this way, EMG contraction can be used to add or multiply existing prosthesis joint angle or torque or other prosthesis parameters during both weight-bearing and non-weight-bearing tasks.

After above-knee amputation, all muscles that control the ankle joint are removed and EMG signals from the muscles of the residual limb do not provide an intuitive way to control the prosthetic ankle. Surgical interventions, such as targeted muscle reinnervation and peripheral nerve interfaces have the potential to provide signals for intuitive control of the prosthesis ankle joint. However, these techniques have not shown the ability to directly control a powered prosthesis during walking, standing up, or other activities. To address this limitation, techniques of the present disclosure combine neural signals and robot control.

In non-amputee individuals, the ankle and knee move in synchrony during standing-up movements, and knee extension is mirrored by ankle plantarflexion. Controller of the present disclosure capture this natural coordination with a linear relationship controlling the equilibrium angle of the prosthesis ankle joint as a function of the measured prosthesis knee angle. This control approach enables the prosthetic foot to lay flat on the ground and support users while they perform many different activities, including standing up, sitting down, squatting, and lunging. The virtual impedance of the ankle joint adds the flexibility necessary to walk in addition to performing standing up activities, without any tuning or calibration of the controller. Because the prosthesis knee position depends on the EMG-controlled prosthesis knee torque, this shared control strategy provides users with indirect volitional control of the prosthesis ankle joint.

By implementing a shared neural controller, as discussed in more detail hereinafter, a user can voluntarily change the torque or position generated by the powered knee prosthesis, or change a parameter within the controller, controlling the timing and amount of energy provided by the powered prosthesis. As a result, the powered prosthesis significantly reduces the amount of compensatory work done by the user's intact and residual limb. Compared to using a conventional passive prosthesis, the disclosed shared neural controller(s) may facilitate significantly reduced muscle effort in both the intact (e.g., 21%-51% decrease in an example implementation) and the residual limb (e.g., 38%-48% decrease in an example implementation). By implementing the disclosed systems, the weight liftable by the prosthesis side increases significantly while standing up with the powered prosthesis (e.g., 49%-68% increase in an example implementation), leading to better loading symmetry (e.g., 43%-46% of body weight on the prosthesis side in an example implementation). Decreased muscle effort is clinically meaningful because muscle fatigue has been linked to increased fall risk. Increased prosthesis loading is clinically meaningful because loading asymmetry is correlated with increased fall risk. In addition, the disclosed shared neural controller(s) may allow for substantial variations in torque, power, and timing, enabling users to stand up from different chairs (e.g., 38-54 cm in example implementations), stand up slower and faster (e.g., 0.5-2.2 sec in example implementations), stand up while carrying a load (e.g., 0-30 lbs. in example implementations), as well as squat, lunge, and walk. Users may also be able to seamlessly transition between activities, which is critical for ambulation in the real world. Additional details related to the foregoing findings are provided hereinafter.

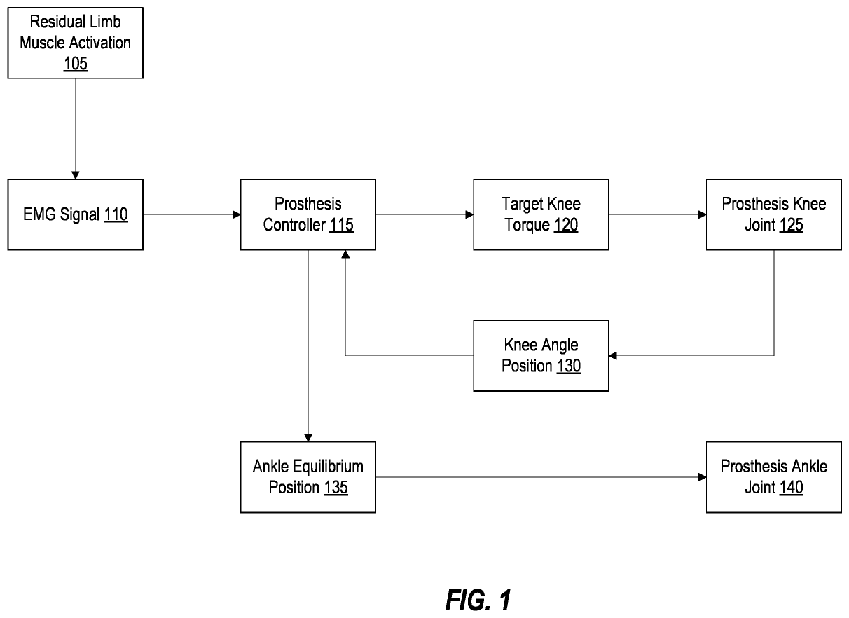

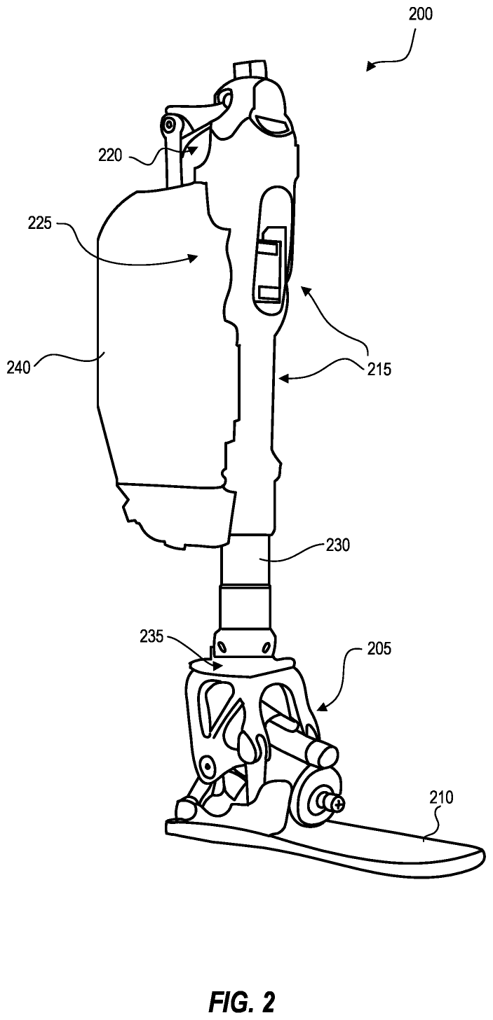

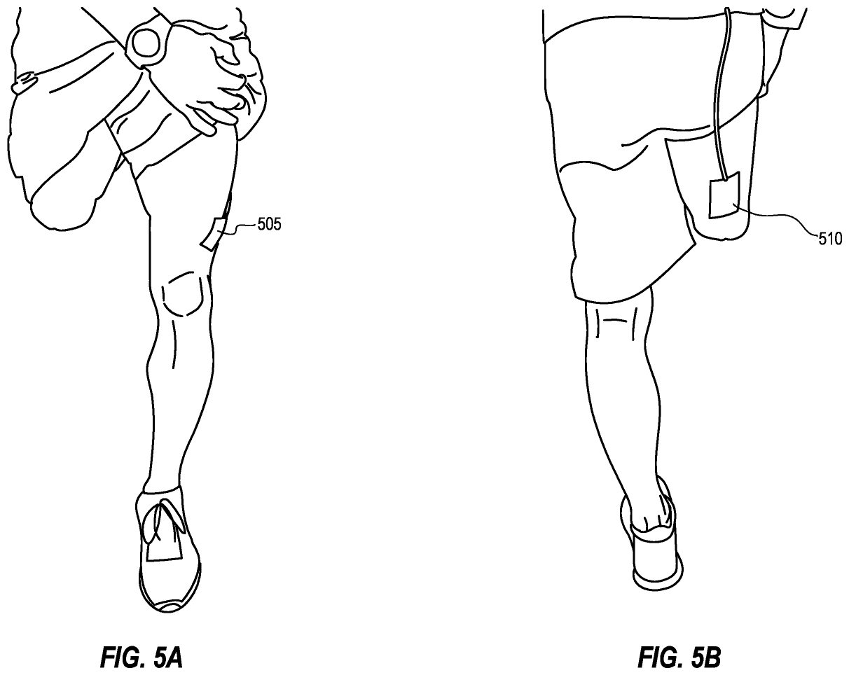

FIG. 1 provides a conceptual representation of operation of a shared neural controller (e.g., in a stance state), in accordance with the present disclosure. FIG. 1 depicts residual limb muscle activation 105, which may comprise muscle activation of a biceps femoris of an above-knee amputee. An EMG sensor may be placed over the skin of the residual limb (see FIG. 5B), thereby generating an EMG signal 110 based on the residual limb muscle activation 105. The EMG signal 110 may be provided as input to a prosthesis controller 115. The prosthesis controller 115 may comprise one or more processing devices that are configured to execute computer-readable instructions to carry out functions/acts related to operating a powered knee and ankle prosthesis (e.g., powered knee and ankle prosthesis 200 of FIG. 2). Based on the input EMG signal 110, the prosthesis controller 115 may generate or determine a target knee torque or position 120 (e.g., in accordance with Equation (1), and/or Equation (3), as described in more detail hereinafter). The target knee torque or position 120 may be used to operate a prosthesis knee joint 125. For instance, a motor may be actuated to apply a torque at the prosthesis knee joint 125 in accordance with the target knee torque or position 120.

The prosthesis knee joint 125 may have a knee angle position 130 associated therewith (e.g., while a torque is being applied at the prosthesis knee joint 125, and/or at other times). The knee angle position 130 may be accessed by the prosthesis controller 115 and used to generate an ankle equilibrium position 135 (e.g., in accordance with Equation (2), as described in more detail hereinafter). The ankle equilibrium position 135 may be used to operate a prosthesis ankle joint 140, such as by causing a motor to actuate into the ankle equilibrium position 135. Such an operational architecture may enable a prosthesis controller 115 to facilitate volitional control of a powered knee and ankle prosthesis (e.g., a prosthesis knee joint 125 and/or a prosthesis ankle joint 140 thereof) without explicit classification of user activity or user intent.

Although the examples discussed in the present disclosure focus, in at least some respects, on shared neural control of a powered joint system implemented as a powered prosthesis (e.g., for above-knee amputees), the principles disclosed herein related to shared neural control may be applied to controllers of other types of powered joint systems, such as powered exoskeleton systems (e.g., powered knee and/or powered ankle exoskeletons that include knee and/or ankle joints).

Having described some of the various high-level features and benefits of the disclosed embodiments, attention will now be directed to FIGS. 2 through 11. These Figures illustrate various supporting illustrations related to the disclosed embodiments.

### Example Powered Knee and Ankle Prosthesis

Systems, methods, and techniques related to shared neural controllers, in accordance with the present disclosure, may be implemented utilizing various types of knee and ankle prostheses. FIG. 2 illustrates a perspective view of an example powered knee and ankle prosthesis 200 that may be implemented in conjunction with the principles disclosed herein related to shared neural controllers. One will appreciate, in view of the present disclosure, that the particular components and/or features of the powered knee and ankle prosthesis 200 of FIG. 2 do not limit the applicability of the disclosed principles related to shared neural controllers to other types of powered knee and ankle prostheses that include additional or alternative components.

The example powered knee and ankle prosthesis 200 of FIG. 2 comprises a self-contained, battery-operated, powered knee and ankle prosthesis that can generate biologically appropriate torque and power during ambulation. The powered knee and ankle prosthesis 200 of FIG. 2 may be configured and/or adjustable to fit users associated with various body sizes. For example, the powered knee and ankle prosthesis 200 may be sized to fit the 50th percentile male leg profile. The powered knee and ankle prosthesis 200 may comprise any suitable weight, such as within a range of about 1.5 kg to about 8 kg (e.g., about 2.5 kg with the battery and protective covers included).

The example powered knee and ankle prosthesis 200 of FIG. 2 comprises an ankle-foot module 205. The ankle-foot module 205 may utilize a compact, lightweight powered polycentric design, which may be contained within a commercially available foot shell. The powered polycentric mechanism of the ankle-foot module 205 may be connected/connectable to custom carbon-fiber feet 210 of different sizes to accommodate different subjects.

The example powered knee and ankle prosthesis 200 of FIG. 2 further comprises a knee module 215. The knee module 215 may utilize an active variable transmission 220 (AVT 220) to optimize the effective transmission ratio and leg dynamics for different locomotion tasks. In addition, the knee module 215 may contain/comprise a control unit and battery 225 and/or motor drivers for both the knee joint and the ankle joint. The knee module 215 and the ankle-foot module may connect with a pylon 230 (e.g., a standard 30-mm pylon), which may allow for height and intra-extra rotation adjustments. In some instances, a pyramid adapter 235 is implemented at the top of the ankle-foot module 205 to estimate the ground reaction force and torque.

The AVT 220 of the example powered knee and ankle prosthesis 200 of FIG. 2 utilizes a DC motor (e.g., a Maxon Motor EC 13, 18 V, 12 W) connected to a 4.1:1 planetary gear, which drives the nut on a bigger, non-backdrivable leadscrew (e.g., M4×1.25, single start) through a 1:1 spur gear transmission. In the example powered knee and ankle prosthesis 200 of FIG. 2, two thrust bearings and two ball bearings are used to support the leadscrew's axial and radial loads. In addition, the lead screw of the AVT 220 can be supported by two parallel guides realized by slotted cranks with dry bushings (e.g., IGUS® Iglidur® L280, static friction coefficient 0.23, dynamic friction coefficient 0.08-0.23). The slotted crank defines the range of motion of the AVT 220 (e.g., a range of motion within a range of about 20 mm to about 45 mm). The overall structural safety factor of the example powered knee and ankle prosthesis 200 represented in FIG. 2 is 2.5. An incremental encoder (e.g., RLS, RM08) is, in some instances, located on the spur gear to measure the position of the AVT 220. This sensor (i.e., the incremental encoder), together with a four-quadrant motor driver (e.g., Maxon Motor ESCON module 24/2) may enable feedback control of the position of the AVT 220 in both driving and braking operations. As noted above, other configurations are within the scope of the present disclosure.

The primary actuator of the example powered knee and ankle prosthesis 200 represented in FIG. 2 is a rotary-to-linear system comprising a brushless DC motor (e.g., Maxon Motor EC-4pole 22, 24 V, 120 W), a roller screw (e.g., Rollvis®, pitch diameter 4.5 mm, lead 2 mm, static-dynamic load ratings 7.2-7.8 kN, efficiency 90%), and a timing-belt transmission (e.g., 48:18 teeth ratio). The roller screw nut is supported by a linear guide (e.g., Helix Linear Technologies, HMR9ML, basic load/moment ratings 3880 N/12.4 Nm). The main motor can be located inside of an aluminium frame (e.g., 7075 T6-SN) which may also operate as a heatsink. A force-torque sensor is, in some instances, embedded in the pylon 230 to detect contact with the ground. Furthermore, in some implementations, a 9-DOF IMU (MPU9250, Invsense) is included to sense the movements and the orientation of the leg in space.

Covers 240 (e.g., 3D printed covers) may be utilized to house the control unit and battery 225. The control unit and battery 225 may comprise a Li-Ion battery (e.g., 2500 mAh, 6S) and/or an onboard system-on-module (SOM) (e.g., myRIO 1900, National Instruments, 100 g without covers). The SOM can run all custom control algorithms in real time, interfacing with the sensors and servo drivers for the AVT 220 and the primary motor (e.g., Elmo, Gold Twitter G-TWI 30/60SE, 35 g). The SOM can be connected through wi-fi to a host computer, smartphone, and/or other device for data monitoring and/or to controller tuning.

Experimental results (discussed in more detail hereinafter) were obtained by implementing a shared neural controller with a powered knee and ankle prosthesis 200 that includes the features/components discussed with reference to FIG. 2. However, as noted previously, the principles discussed herein related to shared neural controllers are not limited to the particular components/features of the powered knee and ankle prosthesis 200 discussed above with reference to FIG. 2.

### Example Shared Neural Control Architecture

In accordance with the present disclosure, at a high-level, a shared neural control architecture for coordinating powered prosthesis movements with human neuromuscular systems may utilize a finite state-machine that comprises two different states—Stance and Swing. In some instances, the Stance state becomes active (or is entered) when it is determined that the prosthesis contacts the ground (e.g., based on detecting a ground reaction force that satisfies a threshold, such as exceeding 50 N). From Stance, the finite-state machine may transition to Swing (e.g., activating the Swing state) when it is determined that the shank position and shank velocity are below thresholds while the knee position is below a threshold. In some implementations, the parameters for the finite-state machine are fixed and do not need to be tuned for different users. In Swing, an indirect volitional controller that is suitable for individuals with above-knee amputation may be utilized, and may be modified by EMG signals. In Stance, shared neural control may be implemented (as discussed hereinabove), such as by utilizing a direct volitional controller based on EMG from the residual limb.

In embodiments, two different low-level controllers are used in Stance for the knee joint (e.g., of knee module 215) and the ankle joint (e.g., of ankle-foot module 205). The knee joint extension torque may be controlled using proportional EMG control, as set forth below in Equation (1).

$$\{ \begin{matrix}{T}_{knee}^{des} = \frac{EMG}{{EMG}_{max}} G \\ G = {G}_{0} + {G}_{1} {θ}_{knee}\end{matrix} \qquad ( 1 )$$

In accordance with Equation (1), the EMG signal from the biceps femoris (EMG) is normalized using its average peak recorded during walking with passive prosthesis (EMGₘₐₓ) and may be multiplied by a position-dependent gain (G) to obtain the target, or desired, knee torque (Tₖₙₑₑ^{des}). The EMG signal may additionally or alternatively be multiplied by a non-position-dependent gain. The passive prosthesis may be achieved by operating a powered prosthesis in a passive mode.

The position-dependent (G) gain can be calculated using a linear curve with an offset (e.g., G₀=30°, or another offset value), as shown in Equation (1). Equation (1) also shows a multiplication factor, G₁, applied to a knee angle position (θₖₙₑₑ). Various multiplication factors may be utilized, such as, by way of non-limiting example, G₁=0.625. When the multiplication factor G₁ is positive, the EMG gain (G) increases with the knee angle position (θₖₙₑₑ), resulting in higher sensitivity of the target torque to the EMG signal for more flexed knee joint angles.

The ankle joint can be controlled using an impedance-based control strategy with fixed or variable stiffness and damping and variable ankle equilibrium position (θₐₙₖₗₑ^{eq}), as set forth below in Equation (2).

$$\{ \begin{matrix}{θ}_{ankle}^{eq} = k {θ}_{knee} & ∀ {θ}_{knee} \geq 0 \\ {θ}_{ankle}^{eq} = 0 & ∀ {θ}_{knee} < 0\end{matrix} \qquad ( 2 )$$

The target ankle equilibrium position (θₐₙₖₗₑ^{eq}) may be configured to change as a function of the measured knee position (θₖₙₑₑ) following the linear relationship shown in Equation (2). The linear relationship between the knee angle position (θₖₙₑₑ) and the target ankle equilibrium position may comprise a negative linear relationship, characterized by negative values of k (e.g., k=−0.133). According to Equation (2), when the knee is fully extended (θₖₙₑₑ=0), the target ankle equilibrium position is set to a neutral standing position (θₐₙₖₗₑ^{eq}=0). When the knee flexes (θₖₙₑₑ≥0), the ankle dorsiflexes (θₐₙₖₗₑ^{eq}<0). Following the Equation (2), and using the example of k=−0.133, the target equilibrium angle of the ankle (θₐₙₖₗₑ^{eq}) reaches a maximum of 12° when the knee joint is flexed at 90°. In the example of Equation (2), the ankle equilibrium position is never positive, so the ankle joint does not actively plantarflex in Stance.

The Stance state may become active in various contexts, such as for standing up, sitting down, squatting, lunging, quiet standing, as well as for the Stance phase of walking, approximately from prosthesis heel-strike to sound side heel-strike, at which point the finite-state machine transitions to Swing, which can operate based on an indirect volitional controller. This indirect volitional controller may be further modified using EMG signals, in order to adjust the prosthesis behavior during transitions between activities, or as desired by the user, according to the following equation:

θₖₙₑₑ=θₖₙₑₑ₁+θ_{kneeEMG}

Or

θₖₙₑₑ=θₖₙₑₑ₁*θ_{kneeEMG} (3)

In this manner, existing controllers for powered prosthesis controller behaviors (θₖₙₑₑ₁) may be modified by adding or multiplying with terms that are proportionally derived from EMG signals (θ_{kneeEMG}) θ, in Equation (3), can be a joint position, joint torque, or even a parameter within the controller, which is modified by the EMG signal. This allows the user to modify the existing behavior of the powered prosthesis according to their needs.

The shared neural control architecture discussed hereinabove may be implemented utilizing a controller (e.g., of control unit and battery 225) of a powered knee and ankle prosthesis (e.g., powered knee and ankle prosthesis 200 of FIG. 2). For example, a controller may be configured in communication with various sensors for obtaining sensor data usable to facilitate control of a powered knee and ankle prosthesis, such as one or more EMG sensors (e.g., to obtain EMG as discussed above with reference to Equation (1)), one or more knee joint angle sensors (e.g., for detecting flexion angle θₖₙₑₑ of a powered knee joint, as discussed above with reference to Equation (1) and Equation (2)), and/or others. The EMG sensor(s) may be placed on skin of the posterior side skin of a user's residual limb so as to capture EMG signals associated with muscle activation of the user's biceps femoris to facilitate shared neural control as discussed hereinabove. Although examples discussed herein focus, in at least some respects, on EMG signals associated with a posterior side of a user's residual limb, the principles discussed herein may utilize EMG signals associated with different parts of a user's body. For example, in some embodiments, the EMG sensor may be placed on additional or alternative portions of a user's body, such as on the anterior, medial, or lateral side of a thigh (e.g., a residual thigh), on a sound, contralateral limb, and/or on a back of a calf.

The controller may be operatively and/or communicatively coupled to motors and/or other actuators of a powered knee and ankle prosthesis to control operation of the powered knee and ankle prosthesis in accordance with target values determined by the controller. For example, responsive to determining a target knee torque Tₖₙₑₑ^{des} as discussed above in accordance with Equation (1), the controller may generate one or more output signals that may cause a motor of a powered knee and ankle prosthesis to apply a torque at a knee joint (e.g., of knee module 215) in accordance with the calculated target knee torque. Similarly, responsive to determining a target ankle equilibrium position θₐₙₖₗₑ^{eq} as discussed above in accordance with Equation (2), the controller may generate one or more output signals that may cause a motor of a powered knee and ankle prosthesis to apply a torque that causes the ankle joint (e.g., of ankle-foot module 205) to assume the target ankle equilibrium position.

The controller may be further configured to detect triggering conditions for determining whether to operate in the Stance state or the Swing state, as discussed above.

### Example Methods

The following discussion now refers to a number of methods and method acts that may be performed in accordance with the present disclosure. Although the method acts are discussed in a certain order and illustrated in a flow chart as occurring in a particular order, no particular ordering is required unless specifically stated, or required because an act is dependent on another act being completed prior to the act being performed. One will appreciate that certain embodiments of the present disclosure may omit one or more of the acts described herein.

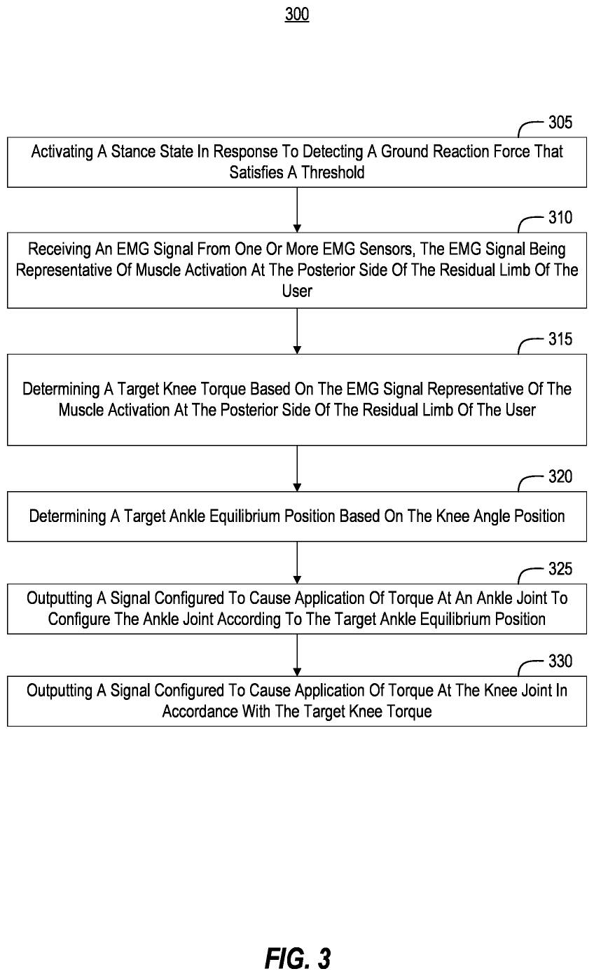

FIG. 3 illustrates an example flow diagram 300 depicting acts associated with providing volitional control of prosthesis joint movement, in accordance with the present disclosure. The acts depicted in flow diagram 300 may be performed utilizing various hardware elements discussed hereinabove, such as controllers (e.g., of control unit and battery 225), sensor(s) (e.g., EMG sensors, knee angle position sensors, etc.), motors, etc. A controller may comprise one or more processing devices and may comprise or access one or more hardware storage devices to facilitate execution of stored instructions to carry out one or more of the acts/functions described herein.

Act 305 of flow diagram 300 includes activating a stance state in response to detecting a ground reaction force that satisfies a threshold. In some instances, act 305 is performed utilizing a pyramid adapter for detecting ground reaction force and/or torque. In some implementations, the stance state is activated based on detecting a ground reaction force that exceeds a threshold of 50 N.

Act 310 of flow diagram 300 includes receiving an EMG signal from one or more EMG sensors, the EMG signal being representative of muscle activation at the posterior side of the residual limb of the user. In some instances, the one or more EMG sensors are adapted for placement on skin of a residual limb of a user to detect EMG signals from the posterior side of the residual limb (see FIG. 5B). The EMG signal can be representative of muscle activation of a biceps femoris of the residual limb of the user. In some embodiments, the EMG sensor may be placed on additional or alternative portions of a user's body, such as on the anterior, medial, or lateral side of a thigh (e.g., a residual thigh), on a sound, contralateral limb, and/or on a back of a calf.

Act 315 of flow diagram 300 includes determining a target knee torque based on the EMG signal representative of the muscle activation at the posterior side of the residual limb of the user. Such functionality may be facilitated utilizing one or more processors of a controller executing stored instructions. In accordance with the present disclosure, the desired knee extension torque can advantageously be determined without explicit classification of user movement or user activity.

In some implementations, determining the target knee torque (e.g., Tₖₙₑₑ^{des} of Equation (1)) includes normalizing the EMG signal (e.g., EMG of Equation (1)) based on an average peak EMG value (e.g., EMGₘₐₓ of Equation (1)). The average peak EMG value can be determined based on measurements associated with the user walking with a passive prosthesis. The passive prosthesis may be one prescribed to a particular user.

The target knee torque can be determined based on a knee angle position such that higher knee target torque is obtained at higher knee angle positions for a same EMG signal. For example, determining the target knee torque may include multiplying the EMG signal by a position-dependent gain (e.g., G of Equation (1)), where the position-dependent gain is based on a knee angle position (e.g., θₖₙₑₑ of Equation (1)) detected by one or more knee angle position sensors. In some instances, the position-dependent gain comprises a product of the knee angle position (e.g., θₖₙₑₑ of Equation (1)) and a multiplication factor (e.g., G₁ of Equation (1)). The product can be further modified by an offset value (e.g., G₀ of Equation (1)).

Act 320 of flow diagram 300 includes determining a target ankle equilibrium position based on the knee angle position. The target ankle equilibrium position may correspond to θₐₙₖₗₑ^{eq}, as discussed hereinabove with reference to Equation (2). In some instances, in response to determining that the knee angle position is greater than or equal to zero, the ankle equilibrium position is defined following a negative linear relationship (e.g., θₐₙₖₗₑ^{eq}=kθₖₙₑₑ, where k is negative) between the knee angle position (e.g., θₖₙₑₑ) and the ankle equilibrium position (θₐₙₖₗₑ^{eq}). In some instances, in response to determining that the knee angle position is less than zero, the ankle equilibrium position is defined as zero.

Act 325 of flow diagram 300 includes outputting a signal configured to cause application of torque at an ankle joint to configure the ankle joint according to the target ankle equilibrium position. The ankle joint may be part of an ankle module (e.g., ankle-foot module 205) of a powered knee and ankle prosthesis (e.g., powered knee and ankle prosthesis 200 of FIG. 2). The signal may be received by a motor of a powered knee and ankle prosthesis to facilitate the actuation of the ankle joint.

Act 330 of flow diagram 300 includes outputting a signal configured to cause application of torque at the knee joint in accordance with the target knee torque. The knee joint may be part of a knee module (e.g., knee module 215) of a powered knee and ankle prosthesis (e.g., powered knee and ankle prosthesis 200 of FIG. 2). The signal may be received by a motor of a powered knee and ankle prosthesis to facilitate the actuation of the knee joint.

### Examples

It shall be noted that these experiments and results are provided by way of illustration and were performed under specific conditions using a specific embodiment or embodiments. Aspects of the experimental protocol(s) discussed below may be applied in real-world and/or end-use contexts (e.g., experimental apparatus(es)/device(s), placement of EMG sensor(s), ambulation activities, and/or others). However, neither these experiments (including the specific experimental conditions or embodiment(s)) nor their results shall be used to limit the scope of the present disclosure.

Participant Information

Two individuals with unilateral above-knee amputation participated in these experiments. Table 1 included below provides demographics related to the participants.

TABLE 1

| Sub- | | Height | Weight | Amputa- | Amputation | Amputation |
|---|---|---|---|---|---|---|
| ject | Age | (m) | (kg) | tion side | cause | years ago |
| S1 | 26 | 1.78 | 64.9 | R | Traumatic | 5 |
| S2 | 30 | 1.60 | 59.0 | L | Traumatic | 10 |

Experimental Protocol

A series of tests were performed by the subjects using a prescribed passive (e.g., their own prescribed passive prosthesis) and a powered knee and ankle prosthesis in under shared neural control (according to the present disclosure). Both subjects performed the tests with their respective prescribed passive prosthesis first. Table 2 provides an overview of activities performed in the experiments.

TABLE 2

| | Passive | Powered |
|---|---|---|
| Activities | Walk | Walk |
| | Sit to stand | Sit-to-stand (regular chair, |
| | (regular chair, | self-selected speed, no weight) |
| | self-selected | Stand-up (tall chair) |
| | speed, no | Stand-up (short chair) |
| | weight) | Stand-up (as fast as possible) |
| | | Stand-up (as slow as possible) |
| | | Stand-up (with weight) |
| | | Stand-up partially |
| | | Squat |
| | | Lunge |
| | | Ambulation circuit |
| | | (Stand-up, walk, |
| | | squat, walk, turn, |
| | | walk, lunge, walk, |
| | | sit-down) |

Walking: subjects walked on level ground at their preferred speed and cadence. A 24-foot walkway allowed for 4-5 consecutive strides. Subjects walked back and forth until at least 20 steady-state strides were recorded (excluding first, last, and turning steps). Subjects performed this walking test with their prescribed passive prosthesis and then a powered prosthesis with the a shared neural controller, as discussed hereinabove.

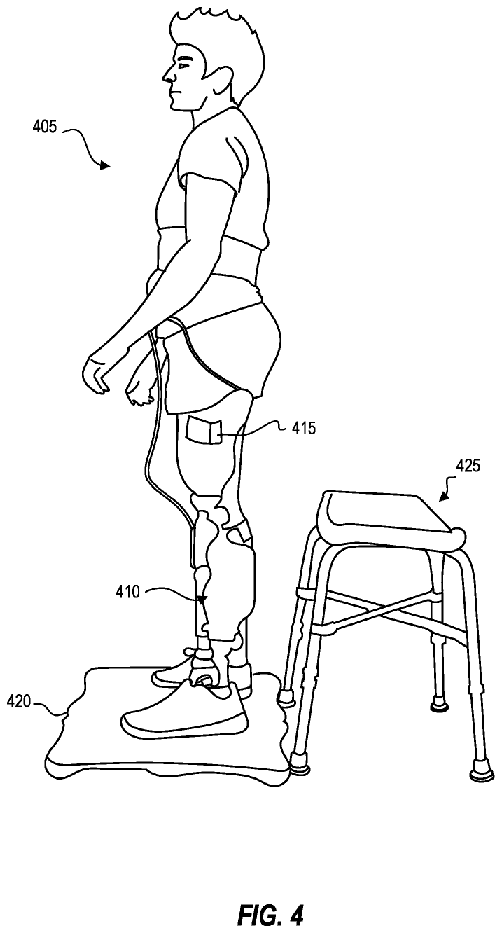

Sit to stand: subjects stood up and sat down from an armless, adjustable-height chair, with each foot placed on a separate force plate (see FIG. 4, which illustrates a subject 405, a powered prosthesis 410, a motion tracker 415, force plates 420, and an adjustable chair 425). For all sit-to-stand transitions, subjects positioned their feet evenly on the force plates. Subjects did not touch the chair with their hands so they could not use their hands to push themselves up. However, subjects were allowed to place their hands on their thighs if needed. Subject performed sit-to-stand transitions at their comfortable speed with their prescribed passive prostheses using a standard chair height of 50 cm (measured from the top of the force plates 420 to the top of the chair seat of the adjustable chair 425). Next, subjects performed sit-to-stand transitions with the powered prosthesis (powered prosthesis 410) under different conditions, selected to simulate real-world conditions. Subjects performed sit-to-stand transitions with three different chair heights—tall, standard, short. The standard chair height was 50 cm—the same used for the prescribed passive prosthesis. The tall chair height was 54 cm (e.g., between the height of a dining chair and a counter chair). The height of the short chair was 42 cm for S1 and 38 cm for S2. This height is similar to that of a standard toilet. The difference in chair height between subjects was due to the range of motion of the powered prosthesis, which was limited by the user's socket for S1.

Subjects also performed partial sit-to-stand transfers using the armless chair set at standard height (50 cm). Specifically, they stood up partway and immediately sat down again, as if they had begun standing up but changed their mind and returned to a seated position. Finally, subjects stood up and sat down from a standard-height chair (50 cm) as quickly as possible and as slowly as possible.

Squat: subjects squatted with the powered prosthesis while holding onto a handrail for safety. Subjects were encouraged to squat as deep as they felt confident and safe. Subjects performed 10-12 repetitions, and the last 6 were used to determine experimental results.

Lunge: subjects lunged with the powered prosthesis in front, while holding onto a side handrail for safety. Subjects placed the prosthesis in front of them and bent both knees to lunge as deep as they felt comfortable, and then stepped through the lunge and took a step before performing another lunge. Subjects performed 10-12 repetitions, and the last 6 were used to determine experimental results.

Ambulation circuit: subjects completed an ambulation circuit with different activities connected by walking. The circuit proceeded as follows: to stand up from a chair, take two steps, squat/lunge, take two steps, turn, take two steps, lunge/squat, take another two steps, turn, and sit down into a chair. 51 subject performed the lunge first and the squat second, and S2 performed the squat first and the lunge second. The entire circuit was performed next to a handrail, and the subjects were told to hold onto it if they felt it was necessary. A chair with arms was used, and subjects were not given specific instructions about whether to use their hands to stand up and sit down.

Experimental Instrumentation and Systems

Motion Analysis System: the subject wore an IMU-based motion tracking system (e.g., MTw Awinda, Xsens, Netherlands) to record kinematic data. Motion trackers were placed on the subject's sternum and lumbar spine, as well as both feet, calves, and thighs. On the amputation side, the thigh motion tracker was attached to the socket (see motion tracker 415 of FIG. 4), and the calf and foot motion trackers were attached to the prosthesis. Body measurements were taken with calipers, and entered into the motion tracking software. The motion tracking system was calibrated while the subject walked. Calibration was performed at the beginning of the session, and after changing between the passive and powered prostheses.

Example Electromyography Sensors

Two surface electromyography sensors (e.g., 13E202=60, Otto Bock, Germany) were placed on the subjects' skin. The first electrode was placed on the subject's intact limb, superficial to a quadricep muscle, the vastus lateralis (as shown in FIG. 5A, illustrating example placement of EMG sensor 505). Electrode placement was determined using standard methods. An area of skin over the muscle belly was shaved and prepped with alcohol. The electrode (e.g., EMG sensor 505) was attached to the subjects' skin using a small square of kinetic tape. The second electrode was placed on the residual limb inside the subject's socket and liner, superficial to a hamstring muscle, the biceps femoris (as shown in FIG. 5B, illustrating example placement of EMG sensor 510). The subject was asked to stand and repeatedly extend their hip against resistance in order to activate the hamstring muscles. The posterior part of the residual limb was palpated to feel for the contracting muscles. The experimentor confirmed that the contracting muscle was a hamstring muscle by following it to its origin on the ischial tuberosity. An area of skin over the muscle belly of the muscle was prepped with alcohol. The residual limb was not shaved, as it can lead to ingrown hairs and irritation within the socket. The EMG electrode (e.g., EMG sensor 510) was placed over the muscle belly and attached to the subject's skin with a square of kinetic tape. The electrode's wire was taped as necessary to prevent it from moving. The subject was asked to extend their hip to check signal quality. The gain dial on the electrode was adjusted until the signal was clear and strong, but not saturating, and the liner and socket were donned over the electrode and wire. The magnitude of the contraction signals was observed during standing up and walking with the subject's passive prosthesis, and the gain dials were adjusted further if necessary.

Force Plates: during sit-to-stand trials, ground reaction forces (GRF) from each foot were recorded using two force plates (e.g., Wii Balance Nintendo; force plates 420 of FIG. 4). GRF data were recorded using a custom LabView program (e.g., National Instruments), and the data from the two force plates were synchronized with each other.

Powered Knee and Ankle Prosthesis: the Utah Lightweight Leg was used for the experimentation. The powered knee and ankle prosthesis 200 shown and described with reference to FIG. 2 is representative of the Utah Lightweight Leg that was used for the experimentation, implementing the particular components and specifications provided by way of example with reference to FIG. 2. The powered prosthesis 410 of FIG. 4 also corresponds to the powered knee and ankle prosthesis 200 of FIG. 2 (i.e., the Utah Lightweight Leg).

Setup and calibration: a certified prosthetist adjusted the height of the powered prosthesis (e.g., powered knee and ankle prosthesis 200, powered prosthesis 410), fit the powered prosthesis to the subject, and ensured proper alignment. The EMG electrodes (e.g., 13E202=60, Ottobock, Germany) were placed on the subject's intact vastus lateralis and residual biceps femoris (see FIGS. 5A and 5B). The subject's liner and socket were placed over the EMG electrode located on the residual biceps femoris muscle (EMG sensor 510). The passive or powered prosthesis was attached to the subject's socket. An IMU-based motion tracking system (e.g., Xsens MVN, Enschede, Netherlands) was attached to the subject's trunk and lower body to measure kinematics. Sensors were placed on the sternum, lumbar spine, intact thigh, intact shank, intact foot, prosthesis socket, prosthesis shank, and prosthesis foot. The motion tracking system was calibrated using the calibration procedure specified by the manufacturer, in which the subjects walked for −20 feet, turned around, and walked back, before stopping and holding a neutral pose. During calibration, the powered prosthesis was controlled with a non-neural walking controller. The calibration was performed at the start of the session and after switching between the prescribed passive prosthesis and the powered prosthesis.

### Experimental Results

As noted above, a single surface EMG electrode (e.g., 13E202=60, Ottobock) was placed on the posterior side of the subjects' residual limbs to measure the activation of a residual hamstring muscle—the biceps femoris (FIG. 5B). After above-knee amputation, the biceps femoris loses its ability to flex the knee and becomes a monoarticular hip extensor. The EMG signal of the biceps femoris is translated into a desired knee extension torque using a position-dependent gain, so that higher torque was obtained at higher knee flexion angles for the same muscle activation (e.g., according to Equation (1)). The equilibrium angle of the powered ankle joint was defined as a linear function of the prosthetic knee position, mimicking the physiological knee-ankle relationship observed in non-amputee individuals during standing up (e.g., according to Equation (2)). The disclosed shared neural controller was implemented in a powered knee and ankle prosthesis (powered knee and ankle prosthesis 200, powered prosthesis 410) and tested by two individuals (S1 and S2) with above-knee amputations, who performed sit-to-stand, squats, lunges, level-ground walking, and transitions between activities.

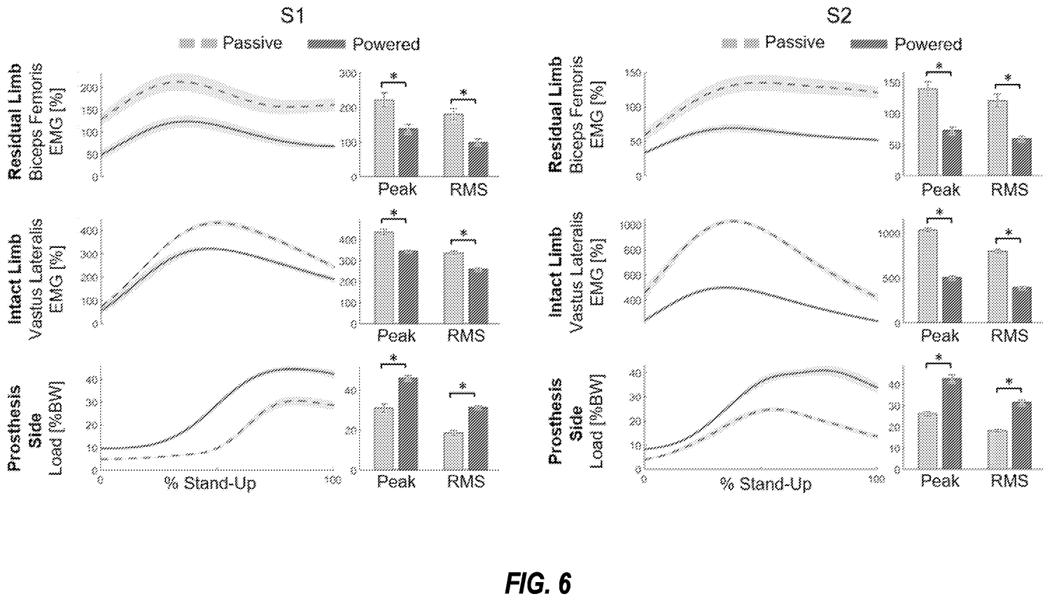

Subjects performed sit-to-stand with the powered prosthesis using the disclosed shared neural controller and with their prescribed passive prostheses while the EMG activations of the residual limb biceps femoris and intact limb vastus lateralis muscles, as well as ground reaction forces, were measured. For both subjects (S1 and S2) and both muscles, the EMG activations were significantly lower with the powered prosthesis than the passive prosthesis, as represented in FIG. 6, which shows comparisons between stand-up with powered prosthesis under the disclosed shared neural controller (dark solid line) vs. standard of care (passive prosthesis) (light grey dashed line) for each subject (S1 and S2). Shaded areas indicate within-subject standard errors. The bar plots show within-subject means (bar) and standard errors (brackets). The asterisks indicate statistically significant difference between prosthesis conditions.

With the powered prosthesis, the peak of the residual biceps femoris EMG was reduced by 38% for S1 and by 48% for S2, and the RMS was reduced by 45% for S1 and 50% for S2. Peak intact vastus lateralis EMG was reduced by 21% for S1 and 51% for S2, and the RMS was reduced by 23% for S1 and 51% for S2. The EMG activations of both the residual biceps femoris and intact vastus lateralis muscle during passive and powered stand-up had different magnitudes but similar patterns, with no significant difference in the timing of the peak activation between conditions (p=0.83 for S1 and p=0.94 for S2). The powered prosthesis lifted significantly more of the subjects' weight during stand-up compared to the passive prostheses (p<0.01). With the powered prosthesis, the peak of the load lifted by the prosthesis increased by 49% for S1 and 63% for S2, whereas the RMS of the load on the prosthesis side increased 68% for S1 and 73% for S2. The resulting peak loading on the prosthesis side was 46.1±4.29% of body weight for S1 and 42.7±6.98% of body weight for S2 during powered stand-up. Thus, the powered prosthesis with the disclosed shared neural controller significantly reduced muscle effort and improved symmetry during standing up, compared to standing up with conventional passive prostheses.

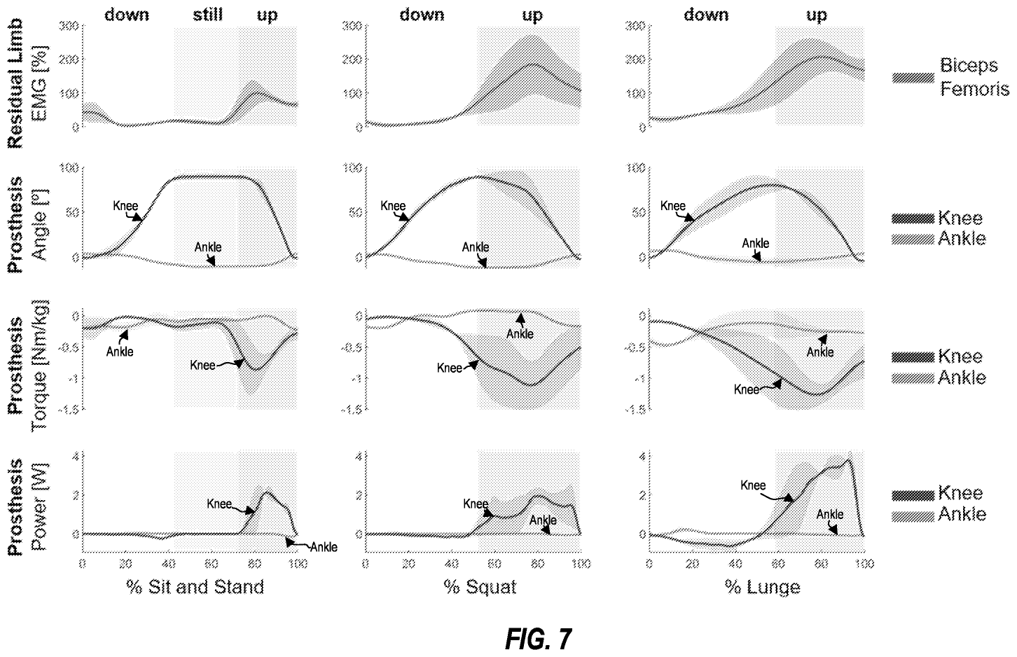

Subjects performed sit-to-stand, squat, and lunge with the powered prosthesis using the disclosed shared neural controller. FIG. 7 shows graphs representing time-series data obtained during these activities. FIG. 7 shows the residual limb biceps femoris EMG signals and the joint angles, joint torques, and joint powers at the prosthesis (black) and the prosthesis ankle joint (grey). The horizontal-axis shading indicates parts of the movement, labeled at the top of the graphs (e.g., “down”, “still”, “up”). The shading along the graph lines indicates between-subject standard errors. As shown in FIG. 7, the average prosthesis knee and ankle joint angles had similar ranges of motion during the three activities. In contrast, the residual biceps femoris EMG activation and the prosthesis knee power showed significant differences between activities for both subjects (p<0.01). Average residual biceps femoris EMG activations peaked at 99±42%, 184±108%, and 206±59%, for sit and stand, squat, and lunge respectively. Average knee power peaked at 2.12±0.31 W during stand-up, at 1.94±0.44 W during squat, and at 3.77±0.30 W during lunge. There were visible differences in knee extension torque, which peaked at −0.87±0.35 Nm/kg, −1.12±0.39 Nm/kg, and −1.27±0.19 Nm/kg for sit and stand, squat, and lunge, respectively (p=0.01 for S1, p<0.01 for S2). Lunge was the fastest activity, with an average knee angular velocity peaking at 149.7±43.6°/sec, compared to 106.2±20.2°/sec for squat and 134.3±3.8°/sec for sit and stand. The torque and power at the ankle joint were consistently smaller than at the knee joint for all the activities.

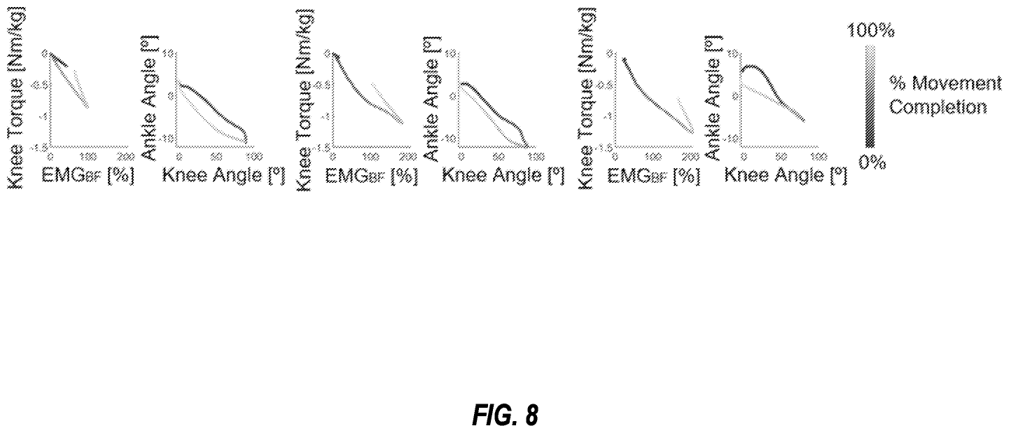

FIG. 8 illustrates graphs indicating analysis of the knee torque as a function of the EMG activation. FIG. 8 shows a non-linear, non-monotonic relationship, where the same knee torque can result from different EMG activations, a result of the position-dependent gain used to compute the desired knee torque. Although the equilibrium angle of the ankle changes linearly with the measured knee angle, the relationship between the measured ankle angle and the measured knee angle shows an elongated circular shape, a result of the low-level impedance control used for the ankle. As indicated by FIGS. 7 and 8, the disclosed shared neural controller enabled the subjects to adapt the prosthesis movements as necessary to perform sit-to-stand, squats, and lunges with the same controller.

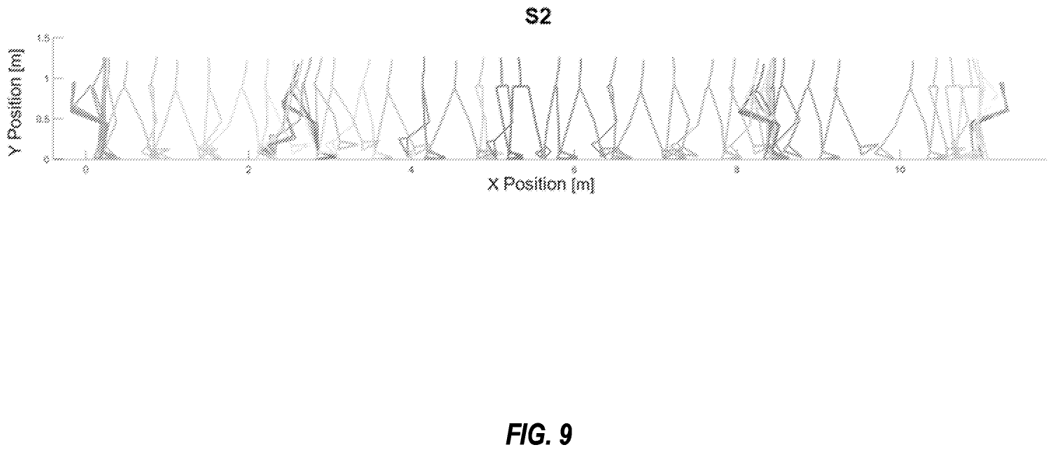

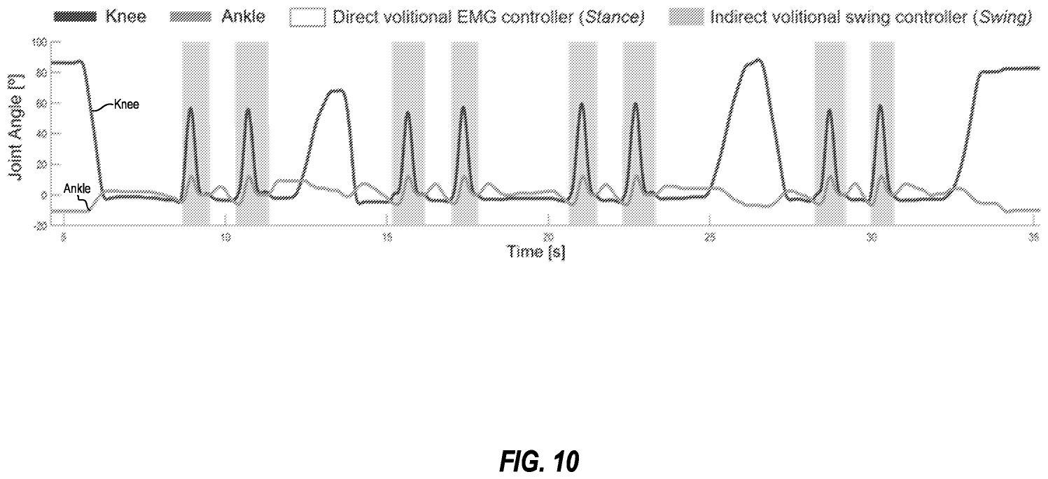

Subjects performed a series of sit-to-stands under different conditions that could be encountered in real life with the powered prosthesis under shared neural control. FIG. 9 illustrates cartesian positions of body segments during a circuit of activities including standing up, walking, squatting, turning, walking, lunging, walking, and sitting down. The cartesian coordinates during the second half of the circuit (after the subject turned around) have been mirrored. FIG. 10 illustrates a graph of the knee and ankle angle during the circuit of activities. Table 2 below provides a

Subjects were able to change their stand-up duration from 0.5 to 2.2 seconds—a 340% difference. Subjects were able to stand up from chairs of different heights, ranging from a minimum of 38 cm (the height of a standard toilet) to a maximum of 54 cm (the height of a tall chair). When subjects stood up from a shorter chair, their EMG activations were significantly higher. Subjects were also able to stand up while wearing a backpack, which resulted in significantly larger prosthesis knee torques and EMG activations compared to standing up without the backpack (p<0.01). Subjects were also able to stand-up partially, as if they had begun standing up and then changed their mind. Compared to normal stand-up, both subjects' knee range of motion decreased significantly during partial stand-up, from 94° to 44° for S1 and from 92° to 49° for S2 (p<0.01). Thus, the disclosed shared neural controller enabled the subjects to change the prosthesis movement as necessary to stand up with different timing, geometry, and loading conditions.

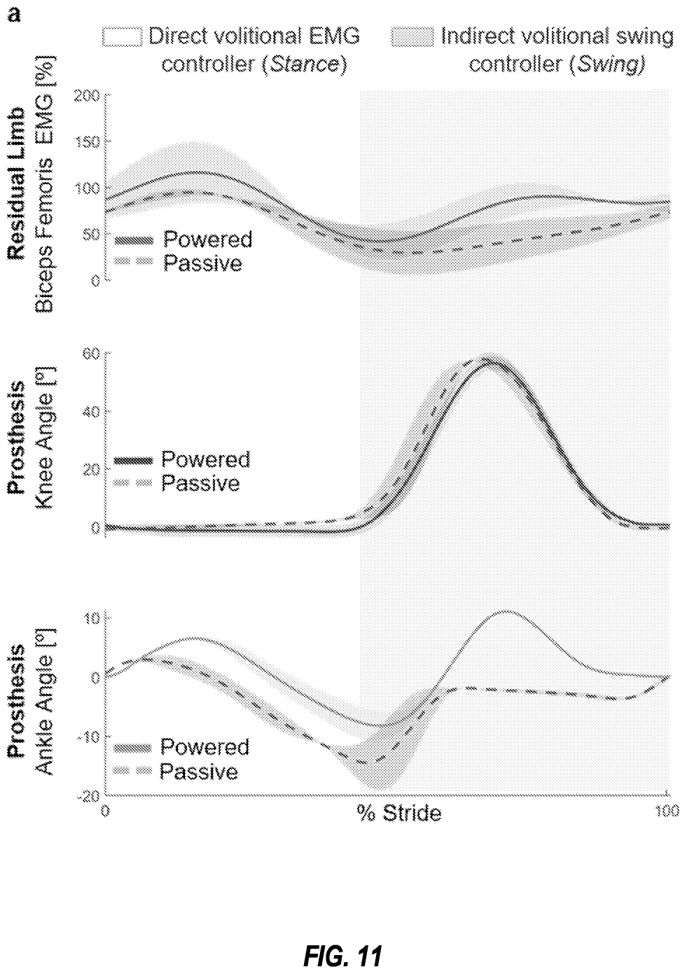

Subjects walked on level ground with both their prescribed passive prosthesis and the powered prosthesis using the disclosed shared neural controller. FIG. 11 illustrates a comparison of walking with the powered knee ankle prosthesis under the disclosed shared neural controller (dark solid line) compared to walking with passive prostheses (light grey dashed line). Shaded areas indicate between-subject standard errors. As shown in FIG. 11, the residual biceps femoris EMG activations were highest during the stance phase of walk (0-50% stride) and had similar patterns and peaks during both passive and powered walking. For both subjects, neither the magnitude nor peak of the residual biceps femoris EMG activations were significantly different between walking with the powered and passive prostheses (peak magnitude: p=0.012 for S1, p=0.066 for S2, peak timing: p=0.219 for S1, p=0.179 for S2). With the shared neural controller, the EMG activation in Stance resulted in knee extension torque that kept the prosthetic knee fully extended while the subject weighted it during stance. Because the knee was fully extended during Stance, the ankle equilibrium angle remained neutral, while the ankle's measured angle changed from plantarflexion (positive) to dorsiflexion (negative), imitating the intact biological ankle kinematics. Thus, walking with the disclosed shared neural controller did not require significant alterations of the residual biceps femoris EMG activations.

Referring again to FIG. 10, as noted above, subjects completed an ambulation circuit in which they stood up, took two steps, squatted, took two steps, turned, took two steps, lunged, took another two steps, turned, and sat down. During the ambulation circuit, the powered prosthesis seamlessly switched between two different states—Stance and Swing. For all ambulation activities, the action of the powered knee and ankle prosthesis during Stance (un-shaded areas in FIG. 10) was controlled directly by the subject's residual biceps femoris EMG (as discussed hereinabove with reference to the shared neural controller architecture), whereas the action of the powered knee and ankle during the Swing (shaded areas in FIG. 10) was controlled using an indirect volitional controller. For both subjects, the ankle and knee angle trajectories did not show discontinuities at the transitions between different activities or controller states (see FIG. 10). The knee kinematics showed noticeable differences during the different activities. The knee angle peaked at 86° in squat, 98° in lunge, and 60.5±0.62° during walk for S1, and 88° in squat, 68° in lunge, and 56.46±0.72° during walk for S2 (see FIG. 10. Subjects were able to seamlessly transition between walking and other activities with the powered prosthesis with the disclosed shared neural controller.

Powered prostheses promise to improve the ambulation ability of millions of individuals with lower-limb amputations. Effective, intuitive, and safe controllers are essential to achieve this goal. Compared to conventional passive prostheses, the standard of care, powered prostheses utilizing the disclosed shared neural controller exhibited a reduction of the compensatory movements necessary to stand up. By putting the user in control of the powered prosthesis, the disclosed shared neural controller enables standing up under a variety of conditions, squatting, lunging, walking, and seamlessly transitioning between activities—none of which are possible with conventional passive prostheses or other powered prosthesis controllers. In the experiments discussed, subjects were able to perform all activities without training, specific instruction, failed attempts, or visual feedback, in contrast with other studies. No subject-specific tuning of the controller was necessary other than adjusting the gain of the EMG sensor as recommended by the manufacturer. Subjects reported that the shared neural controller was easy to use and did not require mental strain or attention.

### Additional Example Aspects

Embodiments of the present disclosure may include, but are not necessarily limited to, features recited in the following clauses:
Clause 1: a powered prosthesis configured to provide volitional control of prosthesis joint movement, the prosthesis comprising: a knee joint; one or more electromyography (EMG) sensors, the one or more EMG sensors being adapted for placement on skin of a residual limb of a user to detect EMG signals from a posterior side of a residual limb; and a controller communicatively coupled to the knee joint and the one or more EMG sensors, the controller comprising one or more processors and one or more hardware storage devices storing instructions that are executable by the one or more processors to configure the controller to: receive an EMG signal from the one or more EMG sensors, the EMG signal being representative of muscle activation at the posterior side of the residual limb of the user; and determine a target knee torque based on the EMG signal representative of the muscle activation at the posterior side of the residual limb of the user.
Clause 2: the powered prosthesis of Clause 1, wherein the EMG signal is representative of muscle activation of a biceps femoris of the residual limb of the user.
Clause 3: the powered prosthesis of Clause 1 or of Clause 2, wherein the target knee torque is determined without explicit classification of user movement or user activity.
Clause 4: the powered prosthesis of any one of Clauses 1 through 3, wherein determining the target knee torque comprises normalizing the EMG signal based on an average peak EMG value.
Clause 5: the powered prosthesis of Clause 4, wherein the average peak EMG value is determined based on measurements associated with the user walking with a passive prosthesis.
Clause 6: the powered prosthesis of any one of Clauses 1 through 5, wherein the target knee torque is determined based on a knee angle position such that higher knee target torque is obtained at higher knee angle positions for a same EMG signal.
Clause 7: the powered prosthesis of Clause 6, wherein determining the target knee torque comprises multiplying the EMG signal by a position-dependent gain.
Clause 8: the powered prosthesis of Clause 7, comprising one or more knee angle position sensors configured to detect the knee angle position associated with the knee joint.
Clause 9: the powered prosthesis of Clause 8, wherein the position-dependent gain is based on the knee angle position detected by the one or more knee angle position sensors.
Clause 10: the powered prosthesis of any one of Clauses 7 through 9, wherein the position-dependent gain comprises a product of the knee angle position and a multiplication factor, the product being modified by an offset value.
Clause 11: the powered prosthesis of any one of Clauses 6 through 11, further comprising an ankle joint, wherein the controller is further configured to control the ankle joint as a function of the knee angle position.
Clause 12: the powered prosthesis of Clause 11, wherein the instructions are executable by the one or more processors to further configure the controller to determine a target ankle equilibrium position based on the knee angle position.
Clause 13: the powered prosthesis of Clause 12, wherein the instructions are executable by the one or more processors to configure the controller to, in response to determining that the knee angle position is greater than or equal to zero, define the ankle equilibrium position following a negative linear relationship between the knee angle position and the ankle equilibrium position.
Clause 14: the powered prosthesis of Clause 12 or of Clause 13, wherein the instructions are executable by the one or more processors to configure the controller to, in response to determining that the knee angle position is less than zero, define the ankle equilibrium position as zero.
Clause 15: the powered prosthesis of any one of Clauses 12 through 14, wherein the instructions are executable by the one or more processors to further configure the controller to output a signal configured to cause application of torque at the ankle joint to configure the ankle joint according to the target ankle equilibrium position.
Clause 16: the powered prosthesis of any one of Clauses 1 through 15, wherein the instructions are executable by the one or more processors to further configure the controller to output a signal configured to cause application of torque at the knee joint in accordance with the target knee torque.
Clause 17: the powered prosthesis of Clause 16, wherein the powered prosthesis is configured to operate in a stance state or in a swing state, and wherein the controller is configured to cause application of torque at the knee joint in accordance with the target knee torque when the stance state is determined to be active.
Clause 18: the powered prosthesis of Clause 17, wherein the powered prosthesis is configured to activate the stance state in response to detecting a ground reaction force that satisfies a threshold.
Clause 19: a method for providing volitional control of prosthesis joint movement, comprising: receiving an EMG signal from one or more EMG sensors, the EMG signal being representative of muscle activation at a posterior side of a residual limb of a user; and determining a target knee torque based on the EMG signal representative of the muscle activation at the posterior side of the residual limb of the user, the target knee torque being determined without explicit classification of user movement or user activity.
Clause 20: one or more hardware storage devices storing instructions that are executable by one or more processors of a controller to configure the controller to: receive an EMG signal from one or more EMG sensors, the EMG signal being representative of muscle activation at a posterior side of a residual limb of a user; and determine a target knee torque based on the EMG signal representative of the muscle activation at the posterior side of the residual limb of the user, the target knee torque being determined without explicit classification of user movement or user activity.

### Additional Terms & Definitions

While certain embodiments of the present disclosure have been described in detail, with reference to specific configurations, parameters, components, elements, etcetera, the descriptions are illustrative and are not to be construed as limiting the scope of the claimed invention.

Furthermore, it should be understood that for any given element of component of a described embodiment, any of the possible alternatives listed for that element or component may generally be used individually or in combination with one another, unless implicitly or explicitly stated otherwise.

In addition, unless otherwise indicated, numbers expressing quantities, constituents, distances, or other measurements used in the specification and claims are to be understood as optionally being modified by the term “about” or its synonyms. When the terms “about,” “approximately,” “substantially,” or the like are used in conjunction with a stated amount, value, or condition, it may be taken to mean an amount, value or condition that deviates by less than 20%, less than 10%, less than 5%, or less than 1% of the stated amount, value, or condition. At the very least, and not as an attempt to limit the application of the doctrine of equivalents to the scope of the claims, each numerical parameter should be construed in light of the number of reported significant digits and by applying ordinary rounding techniques.

Any headings and subheadings used herein are for organizational purposes only and are not meant to be used to limit the scope of the description or the claims.

It will also be noted that, as used in this specification and the appended claims, the singular forms “a,” “an” and “the” do not exclude plural referents unless the context clearly dictates otherwise. Thus, for example, an embodiment referencing a singular referent (e.g., “widget”) may also include two or more such referents.

It will also be appreciated that embodiments described herein may include properties, features (e.g., ingredients, components, members, elements, parts, and/or portions) described in other embodiments described herein. Accordingly, the various features of a given embodiment can be combined with and/or incorporated into other embodiments of the present disclosure. Thus, disclosure of certain features relative to a specific embodiment of the present disclosure should not be construed as limiting application or inclusion of said features to the specific embodiment. Rather, it will be appreciated that other embodiments can also include such features.

What is claimed is:

1. A powered joint system configured to provide volitional control of joint movement, the powered joint system comprising:
a knee joint;
one or more electromyography (EMG) sensors, the one or more EMG sensors being adapted for placement on skin of a limb of a user to detect EMG signals from the limb; and
a controller communicatively coupled to the one or more EMG sensors, the controller comprising one or more processors and one or more hardware storage devices storing instructions that are executable by the one or more processors to configure the controller to:
receive an EMG signal from the one or more EMG sensors, the EMG signal being representative of muscle activation at limb of the user; and
determine a target knee torque or target joint behavior based on the EMG signal representative of the muscle activation at the limb of the user.

2. The powered joint system of claim 1, wherein:
the powered joint system comprises a powered prosthesis;
the limb of the user comprises a residual limb of the user; and
the EMG signal is representative of muscle activation of a biceps femoris of the residual limb of the user.

3. The powered joint system of claim 1, wherein the target knee torque is determined without explicit classification of user movement or user activity, except whether the powered joint system or limb of the user is in contact with the ground.

4. The powered joint system of claim 2, wherein determining the target knee torque comprises normalizing the EMG signal based on an average peak EMG value.

5. The powered joint system of claim 4, wherein the average peak EMG value is determined based on measurements associated with the user walking with a passive prosthesis.

6. The powered joint system of claim 1, wherein the target knee torque is determined based on a knee angle position such that higher knee target torque is obtained at higher knee angle positions for a same EMG signal.

7. The powered joint system of claim 6, wherein determining the target knee torque comprises multiplying the EMG signal by a position-dependent gain.

8. The powered joint system of claim 7, comprising one or more knee angle position sensors configured to detect the knee angle position associated with the knee joint.

9. The powered joint system of claim 8, wherein the position-dependent gain is based on the knee angle position detected by the one or more knee angle position sensors.

10. The powered joint system of claim 7, wherein the position-dependent gain comprises a product of the knee angle position and a multiplication factor, the product being modified by an offset value.

11. The powered joint system of claim 6, further comprising an ankle joint, wherein the controller is further configured to control the ankle joint as a function of the knee angle position.

12. The powered joint system of claim 11, wherein the instructions are executable by the one or more processors to further configure the controller to determine a target ankle equilibrium position based on the knee angle position.

13. The powered joint system of claim 12, wherein the instructions are executable by the one or more processors to configure the controller to, in response to determining that the knee angle position is greater than or equal to zero, define the ankle equilibrium position following a negative linear relationship between the knee angle position and the ankle equilibrium position.

14. The powered joint system of claim 12, wherein the instructions are executable by the one or more processors to configure the controller to, in response to determining that the knee angle position is less than zero, define the ankle equilibrium position as zero.

15. The powered joint system of claim 12, wherein the instructions are executable by the one or more processors to further configure the controller to output a second signal configured to cause application of torque at the ankle joint to configure the ankle joint according to the target ankle equilibrium position.

16. The powered joint system of claim 1, wherein the instructions are executable by the one or more processors to further configure the controller to output a signal configured to cause application of torque at the knee joint in accordance with the target knee torque.

17. The powered joint system of claim 16, wherein the powered joint system is configured to operate in a stance state or in a swing state, and wherein the controller is configured to cause application of torque at the knee joint in accordance with the target knee torque when the stance state is determined to be active.

18. The powered joint system of claim 17, wherein the powered joint system is configured to activate the stance state in response to detecting a ground reaction force that satisfies a threshold.

19. A method for providing volitional control of joint system joint movement, comprising:
receiving an EMG signal from one or more EMG sensors, the EMG signal being representative of muscle activation at a posterior side of a residual limb of a user; and
determining a target knee torque based on the EMG signal representative of the muscle activation at the posterior side of the residual limb of the user, the target knee torque being determined without explicit classification of user movement or user activity.

20. One or more hardware storage devices storing instructions that are executable by one or more processors of a controller to configure the controller to:
receive an EMG signal from one or more EMG sensors, the EMG signal being representative of muscle activation at a posterior side of a residual limb of a user; and
determine a target knee torque based on the EMG signal representative of the muscle activation at the posterior side of the residual limb of the user, the target knee torque being determined without explicit classification of user movement or user activity.

21. A method for modifying existing control of powered joint movement, comprising:
receiving an EMG signal from one or more EMG sensors, the EMG signal being representative of muscle activation of a residual limb of a user;
determining a target joint behavior, the target joint behavior being selected from the group consisting of joint position, joint torque, a value which is multiplied to a desired output, or a parameter within the controller; and
modifying the target joint behavior based on the EMG signal representative of the muscle activation from the residual limb of the user, the target joint behavior being modified without explicit classification of user movement or user activity.
